(19) United States
(12) Patent Application Publication
(10) Pub. No.: US 2026/0124430 A1
(43) Pub. Date: May 7, 2026

(54) Systems and Methods for Providing Variable Support Designed to Aid in Navigation and Delivery of Neurovascular Treatment Instruments to a Treatment Site

(71) Applicant: Unity Medical Inc., Edina, MN (US)

(72) Inventors: Matthew F. Ogle, Edina, MN (US); Brian Packard, Monticello, MN (US); Jacob Joseph Kent, Albertville, MN (US); Bryan Claseman, Buffalo, MN (US)

(73) Assignee: Unity Medical Inc., Edina, MN (US)

(21) Appl. No.: 19/379,233

(22) Filed: Nov. 4, 2025

Related U.S. Application Data
(60) Provisional application No. 63/716,854, filed on Nov. 6, 2024.
(60) Provisional application No. 63/758,524, filed on Feb. 14, 2025.

(51) Int. Cl. A61M 25/01; A61M 25/00; A61M 29/02

(52) U.S. Cl. CPC A61M 25/0102; A61M 25/0013; A61M 25/0108; A61M 29/02; A61M 2029/025

(57) ABSTRACT

In an illustrative embodiment, a support instrument configured to provide additional stiffness to a delivery instrument during advancement of the delivery instrument to a procedural site includes a shaft defining, along at least a portion of its length, a partially enclosed lumen configured to receive at least a portion of the delivery instrument, and radiopaque marker(s) disposed along the length of the shaft and configured to identify an orientation of an opening of the partially enclosed lumen. A distal portion of the shaft may have a support instrument deflection force greater than a delivery instrument deflection force of a distal portion of the delivery instrument. The partially enclosed lumen may be configured to enable advancement of the delivery instrument while the support instrument remains in a same position or is withdrawn.

### RELATED APPLICATIONS

The present application claims the benefit of both U.S. Provisional Patent Application Ser. No. 63/758,524 entitled “Systems and Methods for Providing Variable Support Designed to Aid in Navigation and Delivery of Neurovascular Treatment Instruments to a Treatment Site” and filed Feb. 14, 2025, and U.S. Provisional Patent Application Ser. No. 63/716,854 entitled “Flexible Delivery Catheters for Reduced Vascular Trauma” and filed Nov. 6, 2024. The present application is related to U.S. patent application Ser. No. 18/955,284 entitled “Versatile Delivery Catheter” and filed Nov. 21, 2024. All above-noted applications are hereby incorporated by reference in their entireties.

### BACKGROUND

Current delivery catheters often face challenges related to navigating through the vasculature, especially in sensitive or tortuous areas. Catheters that are overly flexible may backtrack or cause complications during procedures, particularly when advancing through difficult anatomical areas like the aortic arch. Catheters with straight, blunt, or rigid tips may cause damage or excessive trauma to the arterial walls or become caught in small side branches. These issues can extend procedure time and increase the risk of patient complications. A design that minimizes such risks while maintaining effective delivery capabilities is highly sought after in vascular medicine.

Traditional catheters used for stent retrieval and/or delivery often face challenges related to flexibility, lubricity, and navigation through the arterial system. Existing designs may lack the necessary flexibility to maneuver effectively and may not provide adequate lubrication throughout the catheter's length, which can impede performance and increase procedural risks.

### SUMMARY OF ILLUSTRATIVE EMBODIMENTS

In one aspect, the present disclosure relates to a delivery catheter apparatus including a delivery catheter, a flexible shaft, and a support instrument configured to advance coaxially between the flexible shaft and the catheter while the flexible shaft is being advanced to a delivery site to provide varying levels of stiffness depending upon anatomical pathways encountered en route. To best align with the anatomy of a treatment path while navigating without complication, different levels of stiffness may be desired. The support instrument, for example, may include a support sleeve providing a movable section of increased rigidity along a length of a catheter or guide wire. The support instrument, for example, may be positioned in an area of increased angularity to bridge the region of the catheter or guide wire to avoid prolapsing of the catheter or guide wire while traversing the tortuous path.

In one aspect, the present disclosure relates to methods and manufacturing techniques for producing a highly flexible catheter shaft designed to navigate through complex vascular structures with ease. Vascular angularity is difficult to traverse with catheters or wires. In some embodiments, the catheter shaft is enhanced for increased flexibility. In particular, a length of the catheter shaft may be adjusted for increased flexibility in the direction of a curve or shape. The length of the catheter shaft may be proximate to and/or including the catheter tip.

In one aspect, the present disclosure relates to a novel shaped design for flexible delivery catheters featuring a curved tip. The design aims to enhance the catheter's ability to navigate complex vascular pathways while minimizing the risk of injury to the arterial walls. By incorporating a shaping to the tip that facilitates smoother entry and reduced trauma, embodiments of flexible delivery catheter designs described herein represent a significant advancement in catheter technology. The tip, for example, may be designed with a curvature to facilitate smoother entry into the vasculature, reducing the risk of damaging arterial walls. In one example, tip may further include a gradual taper to a pointed end. In another example, the tip may further include a widened (e.g., rounded) end.

Catheters and catheter delivery systems described herein provide many benefits. First, certain embodiments eliminate the need to use a larger sheath for extra support. The smaller diameter delivery system enabled through eliminating the sheath, in some examples, supports a greater number of access sites and provides the opportunity to treat smaller patients. Further, eliminating the sheath reducing the complexity and size of the equipment used.

Catheters and catheter delivery systems described herein may minimize the potential for complications when traversing complex anatomical pathways such as, in some examples, a saccular aneurism, a fusiform aneurism, coronary pathways, and peripheral vascular pathways.

In one aspect, the present disclosure relates to a support instrument configured to provide additional stiffness to a delivery instrument during advancement of the delivery instrument to a procedural site. The support instrument may include a shaft defining, along at least a portion of a length of the shaft, a partially enclosed lumen configured to receive at least a portion of the delivery instrument. A distal portion of the shaft may have a support instrument deflection force greater than a delivery instrument deflection force of a distal portion of the delivery instrument, and the partially enclosed lumen may be configured to enable advancement of the delivery instrument while the support instrument remains in a same position or is withdrawn. The support instrument may include at least one radiopaque marker disposed along the length of the shaft, where the at least one radiopaque marker is configured to identify an orientation of an opening of the partially enclosed lumen.

In some embodiments, the opening of the partially enclosed lumen defines a uniform gap. The shaft may define, along a second portion of the length of the shaft, a fully enclosed lumen configured to receive another portion of the delivery instrument. At least a distal portion of the shaft may include a flexible structure formed from medical grade filaments or fibers. A bottom radiopaque marker of the at least one radiopaque marker may be disposed on a surface of the shaft opposite the opening. The opening may be configured, at least at a proximal end of the shaft, to allow a section of the delivery instrument to be withdrawn at an angle to the length of the shaft, thereby locking a position of the delivery instrument relative to the support instrument. The opening may include a wavy or toothed pattern.

In some embodiments, a material composition of the support instrument includes one or more of a hypotube, stainless steel, nitinol, or carbon fiber. A material composition of the support instrument may include one or more of nylon, polyether block amide (PEBA), polyurethane (PU), thermoplastic polyurethane (TPU), polyether ether ketone (PEEK), polyethylene terephthalate (PET), or polyethylene naphthalate (PEN). The support instrument may include a tapered distal tip.

In one aspect, a method for using a support element to assist in navigating an extending element through a tortuous path includes providing the support element including a shaft, positioning at least a segment of the extending element along an inner curvature of the shaft of the support element, advancing the support element and the extending element together along the tortuous path to align a section of the support element with the tortuous path, and continuing to advance the extending element through the tortuous path to deliver a distal portion of the extending element beyond a distal end of the support element through a section of vasculature distal to the tortuous path.

In some embodiments, positioning the support element includes positioning the support element within a catheter and extending along at least a portion of a length of the catheter. The extending element may include a tapered section spanning a first length of the extending element. The support element may include a complementary thickened section spanning a second length of the support element shorter than or a same length as the first length, where, upon positioning the support element within the catheter, the thickened section of the support element aligns with at least a section of the first length of the extending element, thereby linearly displacing the first length of the extending element to align closer to a wall of the catheter.

In some embodiments, the support element defines, along portions of a length of the shaft, at least one of a partially enclosed lumen or a fully enclosed lumen. The method may include, prior to continuing to advance the extending element, anchoring the support element relative to the extending element to maintain a current position of the support element. The support element may include, along portions of a length of the shaft, at least one radiopaque element, and aligning the section of the support element with the tortuous path may include aligning the at least one radiopaque element with the tortuous path.

In one aspect, the present disclosure relates to a method for manufacturing a catheter with a pre-formed shaped tip, including positioning a lumen of a catheter material on a manufacturing mandrel, and applying a respective one or more cuts along each respective segment of at least one segment of the catheter material proximal to a distal end of the catheter material, the respective segment spanning a length of at least three millimeters, each cut of the respective one or more cuts having a respective depth spanning at least half way into a thickness of a wall of the catheter material along the respective segment. The respective one or more cuts may be applied to a respective portion of a diameter of the catheter material, thereby encouraging directional flexibility of the respective segment to compress into a curve along the respective portion of the diameter. Applying the respective one or more cuts along each respective segment of the at least one segment of the catheter material may result in the distal end of the catheter material being oriented at an angle to the length of the catheter material.

In some embodiments, the at least one segment includes two segments, the respective one or more cuts applied to a first segment of the two segments encourages directional flexibility in a first direction, and the respective one or more cuts applied to a second segment of the two segments encourages directional flexibility in a second direction opposite the first direction, such that the first segment and the second segment create an “S”-shaped curve in the catheter material. A set of curves of the “S”-shaped curve may be formed and sized to align, generally, with a path through a branch of an aortic arch into a carotid artery of a patient.

In some embodiments, the respective one or more cuts applied to the at least one segment create a “U”-shaped curve, such that the distal end of the catheter material is oriented at an angle generally directed toward a proximal end of the catheter material. A curvature of the “U”-shaped curve may be formed and sized to align, generally, with an aortic arch of a patient.

In some embodiments, the distal end of the catheter tapers in diameter from a diameter of each respective segment. Positioning the lumen of the catheter material on the manufacturing mandrel may include positioning a liner material on the manufacturing mandrel, and positioning an outer material on the liner material. Applying the respective one or more cuts along each respective segment of the at least one segment of the catheter material may include applying the respective one or more cuts through the outer material and into the liner material. The method may include, after applying the respective one or more cuts along each respective segment of the at least one segment of the catheter material, heating the manufacturing mandrel, thereby causing the outer material to flow into the cuts made in the liner material.

In some embodiments, the respective one or more cuts along each respective segment of the at least one segment of the catheter material are aligned perpendicular to the length of the catheter material. A respective distance between respective cuts of the one or more cuts along one or more segments of the at least one segment may differ, such that a density of cuts is greatest away from each edge of each segment of the one or more segments.

In one aspect, the present disclosure relates to a method for manufacturing a catheter with a segmented liner, including positioning an inner catheter material layer around a manufacturing mandrel, positioning an outer catheter material layer around the inner catheter material layer on the manufacturing mandrel, heating the outer catheter material layer and the inner catheter material layer on the manufacturing mandrel to reflow at least the outer catheter material layer, thereby creating a bond between the outer catheter material layer and the inner catheter material layer, applying one or more cuts through the outer catheter material layer and the inner catheter material layer along at least a portion of a length of the inner and outer catheter material layers, and after applying the one or more cuts, reheating the outer catheter material layer and the inner catheter material layer on the manufacturing mandrel, thereby causing the outer catheter material layer to flow into the one or more cuts made through the inner catheter material layer.

In one aspect, the present disclosure relates to a flexible catheter, including an inner catheter material including at least one cut, where the at least one cut is formed along at least a portion of a length of the inner catheter material, and an outer catheter material surrounding the inner catheter material and bonded to the inner catheter material. The outer catheter material may be bonded with the inner catheter material in part by heating the outer catheter material and causing the outer catheter material to flow into at least partially into the at least one cut.

In some embodiments, the inner catheter material includes a first melt flow index and the outer catheter material includes a second melt flow index lower than the first melt flow index. The inner catheter material may be composed of at least one of PTFE, PU, PE, polyamide, PEEK, or fluoropolymer. The outer catheter material may be composed of a thermoplastic elastomer. The at least one cut may include at least one spiral cut.

In some embodiments, the at least one cut includes multiple cuts. At least a portion of the multiple cuts may be aligned perpendicular to a length of the flexible catheter. The multiple cuts may include a first set of cuts along a first length of the flexible catheter and a second set of cuts along a second length of the flexible catheter separated from the first length of the flexible catheter. A spacing between pairs of cuts of the multiple cuts may vary along the portion of the length of the inner catheter material.

In some embodiments, an inner diameter of the flexible catheter includes a texturized surface. The at least one cut may provide the texturized surface. A distal length of the flexible catheter proximate a distal tip of the catheter may include a curved shape. The at least one cut may enable the tip to take a form of the curved shape. The curved shape may be an “S”-shaped curve.

In one aspect, the present disclosure relates to a method for dilating an occlusion in vasculature of a patient, including providing a flexible delivery element including a central lumen, and a tip region including a pre-formed angle, where the pre-formed angle causes a distal tip of the flexible delivery element to be offset from a longitudinal axis of the flexible delivery element by up to forty-five degrees. The method may include advancing the flexible delivery element within the vasculature of the patient to deliver the distal tip of the flexible delivery element to a procedural site, and advancing the distal tip of the flexible delivery element into a region of an occlusion at the procedural site, thereby causing dilation of the occlusion.

In some embodiments, the method includes advancing a dilation device within the lumen of the flexible delivery element, and after causing dilation of the occlusion, retracting the distal tip of the flexible delivery element, thereby releasing the dilation device. The dilation device may be a stent.

In some embodiments, the method includes, prior to advancing the distal tip of the flexible delivery element into the region of the occlusion, advancing a length of the flexible delivery element through a challenging anatomical region. The challenging anatomical region may include at least one of an aortic arch, a saccular aneurysm, a fusiform aneurysm, or a brachiocephalic vein. Advancing the flexible delivery element within the vasculature may include advancing the flexible delivery element within a guide element. Advancing the length of the flexible delivery element through the challenging anatomical region may include coaxially introducing a support element between the guide element and the flexible delivery element, and during advancement through the challenging anatomical region, selectively positioning the support element along a distal portion of the flexible delivery element to provide increased stiffness to guide the distal tip of the flexible delivery element in navigating the challenging anatomical region. The support element may include a support element minimum inner diameter configured to receive the flexible delivery element. A support element length of the support element may be shorter than a delivery element length of the flexible delivery element.

In some embodiments, the tip region of the flexible delivery element has a tip flexibility having a lower deflection force than a remainder of the flexible delivery element.

In one aspect, the present disclosure relates to a kit for delivering instrumentation to a treatment site in a patient, the kit including an extending element including a flexible tip, and a support element including a support element minimum inner diameter configured to receive the flexible extending element. The kit may include printed instructions instructing advancing the extending element through a catheter into a vascular pathway, coaxially introducing the support element between the catheter and the extending element, and during the advancing, selectively positioning the support element along a distal portion of the extending element to provide increased stiffness to guide the flexible tip of the extending element in navigating an access path through a vascular system of the patient.

In some embodiments, the extending element is a guide wire or a microcatheter. A support element length of the support element may be shorter than an extending element length of the extending element. The support element minimum inner diameter may be within a range of 0.3 millimeters to 5.3 millimeters. The flexible tip of the extending element may have a tip flexibility having a lower deflection force than a remainder of the extending element. The access path may traverse at least one of an aortic arch, a saccular aneurysm, a fusiform aneurysm, or a brachiocephalic vein. The support element may include one or more radiopaque markers. Selectively positioning may include aligning the one or more radiopaque markers at the distal portion of the extending element.

In some embodiments, the support element includes a flexible material structure including one of a coil configuration, a weaving configuration, or a braiding configuration. The flexible material structure may be imparted by cutting or grinding a lumen composed of rigid or semi-rigid material. The kit may include a catheter including a catheter minimum inner diameter. The support element may include a support element maximum outer diameter configured to fit within the catheter minimum inner diameter.

The foregoing general description of the illustrative embodiments and the following detailed description thereof provide mere examples of various aspects of the teachings of this disclosure and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. In the drawings:

FIG. 1A is a block diagram of an example apparatus for delivering instrumentation to a treatment site;

FIG. 1B illustrates the apparatus of FIG. 1A in assembled form;

FIG. 2A through FIG. 2E illustrate example material structures for a support element;

FIG. 3A through FIG. 3D illustrate example partially open forms for support element structures;

FIG. 4 illustrates an example mechanism for anchoring a support element after positioning along a tortuous pathway;

FIG. 5 illustrates a flow chart of an example method for using a support element to assist in navigating an extending element through a tortuous path;

FIGS. 6A and 6B illustrate an example tortuous path assisted in navigation by an example support element;

FIG. 7A through FIG. 7C illustrate an example curved support element and use therefor;

FIG. 8A and FIG. 8B illustrate radiopaque marker configurations and locations for use with an open or partially open support element;

FIGS. 9A, 9B, and 10A illustrate example shaped tip designs for a flexible extending element;

FIG. 10B illustrates an example use scenario for the example shaped tip design of FIG. 10A;

FIGS. 11A, 11B, and 12A illustrate example liner segmentation patterns;

FIG. 12B illustrates an example scenario for using a catheter with a segmented liner;

FIG. 13 illustrates a first example manufacturing method for producing a catheter with a segmented liner;

FIG. 14 is a flow chart of a second example manufacturing method for producing a catheter with a segmented liner;

FIG. 15A through FIG. 15D illustrate steps of the second example manufacturing method of FIG. 14;

FIG. 16 illustrates an example textured surface on an inner diameter of an example catheter liner; and

FIG. 17 is a flow chart of an example method for dilating a chronic occlusion.

### DETAILED DESCRIPTION OF ILLUSTRATIVE EMBODIMENTS

The description set forth below in connection with the coordinating drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

As used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Further, the terms “approximately,” “about,” “proximate,” “minor variation,” and similar terms generally refer to ranges that include the identified value within some margin, such as, in some examples, 20%, 10%, or 5% in certain embodiments, as well as any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

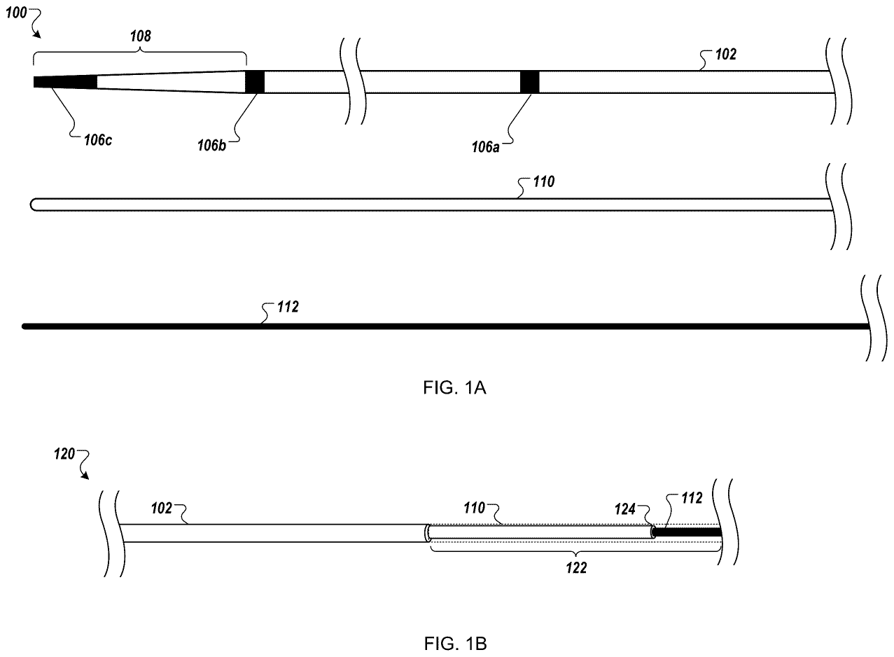

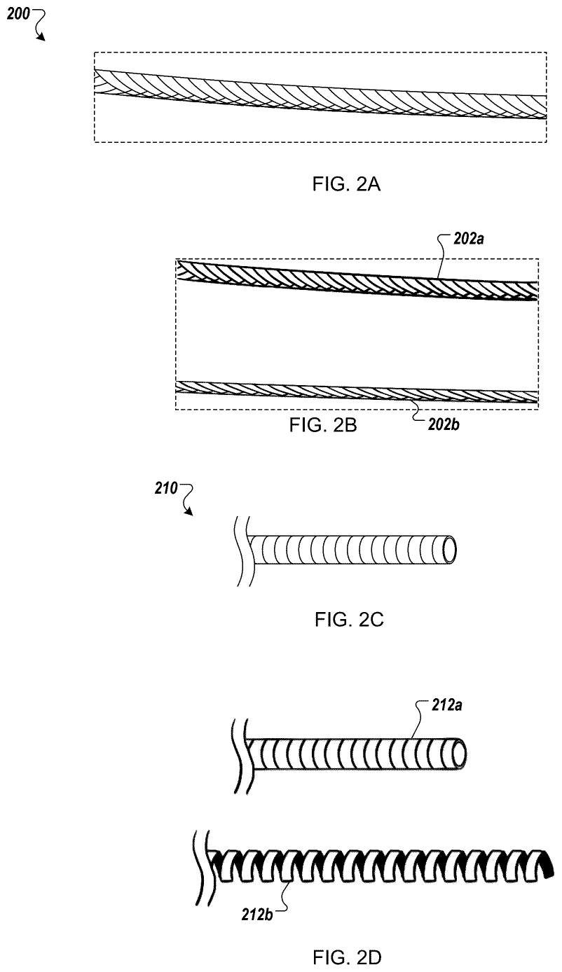

FIG. 1A is a block diagram of an example apparatus 100 for delivering instrumentation to a treatment site. As illustrated, the apparatus includes a guide catheter 102 (e.g., catheter or microcatheter), a support element 110, and a flexible extending element 112 (e.g., delivery catheter, guide wire, stent retriever, etc.). The flexible extending element 112 may be guided to a treatment site via the guide catheter 102. Radiopaque markers 106a-c may be disposed on a surface of the guide catheter 102 so that imaging can track the progress of the guide catheter 102 to the treatment site. The support element 110 may be selectively used during advancement of the flexible extending element 112 to assist in traversing challenging anatomy. For example, the support element 110 may be advanced to and positioned along a challenging portion of the delivery path. When in the desired location, in some embodiments, the support element 110 may be anchored in position while the flexible extending element 112 is advanced through the challenging portion of the delivery path. The challenging anatomy may include various neurovascular, cardiovascular, and/or peripheral access paths such as, in some examples, the aortic arch, a saccular aneurysm, a fusiform aneurysm, or the brachiocephalic veins.

The guide catheter 102, in some embodiments, is a conventional catheter designed for traversing vasculature during a complex interventional medical procedure, such as, in some examples, neurovascular stroke therapy (thrombectomy), aneurysm, angioplasty, and/or stenting. In some embodiments, the guide catheter 102 is designed with increased flexibility to assist in traversing tortuous paths, such as various catheter designs discussed below. The guide catheter 102, for example, may include at least one lumen extending therethrough. The dimensions of the shaft of the guide catheter 102, in some examples, can be about 100 cm to 200 cm in length, or preferably about 120-170 cm in length, with a diameter within a range from 2 to 12 French (e.g., 0.6 mm to 4.0 mm). In a particular example, the shaft of the guide catheter 102 may be 140 cm long and 7 Fr in diameter. As described in greater detail below, regions of the guide catheter 102 may vary in interior and/or exterior diameter. Although illustrated as having a tapered tip 108, in other embodiments, the guide catheter 102 may have a single outer diameter or multiple different (tapered, stepped, and/or widened/rounded) outer diameters.

In some embodiments, to assist in traversing challenging anatomy, the support element 110 is inserted within the guide catheter 102 and over the flexible extending element 112. The support element 110 may be advanced coaxially between the guide catheter 102 and the flexible extending element 112 to provide increased stiffness at a section of the delivery path where the guide catheter 102 and/or flexible extending element 112 may buckle, backtrack, or otherwise veer off course during navigation due to their enhanced flexibility. In another example, the flexible extending element 112 may be advanced coaxially within the guide catheter 102 along with the flexible extending element 112 to a region of challenging anatomy. The support element 110, for example, may be shorter or a similar length as the guide catheter 102. The support element, in some examples, can be 50 cm to 100 cm in length, 100 cm to 200 cm in length, or preferably about 100 cm in length, with a diameter within a range from 1 to 16 French (e.g., 0.3 mm to 5.3 mm). In a particular example, the shaft of the support element 110 may be 70 cm long and six Fr in diameter.

Turning to FIG. 1B, in a partial cutaway view of an assembly 120 of the guide catheter 102, the support element 110, and the flexible extending element 112, as shown in a cutaway section 122, the support element 110 is positioned between a wall of the guide catheter 102 and the flexible extending element 112, while the flexible extending element 112 has been advanced beyond a distal end 124 of the support element 110.

In some embodiments, the support element 110 is positioned about the flexible extending element 112 by pushing the proximal shaft of the flexible extending element 112 into the distal end of the support element 110 (“back loading”). In certain embodiments, the support element is positioned about the flexible extending element 112 by pushing a distal end of the flexible extending element 112 into the proximal end of the support element 110 (“front loading”). In further embodiments, described in greater detail below, the support element 110 is partially open such that it can be introduced onto a length of the flexible extending element 112 without necessarily having access to one of the ends of the flexible extending element 112.

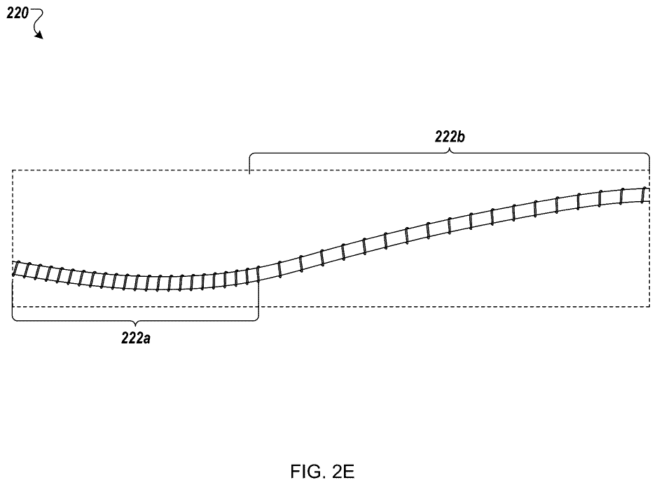

In some implementations, a structure of the material of a support element such as the support element 112 of FIG. 1A and FIG. 1B is formed in a manner that imparts flexing and/or stretching/compacting capabilities to the support element. Material structures for a support element can include, in some examples, various braiding configurations, weaving configurations, and/or coil configurations. To avoid pinching or snagging of instruments inserted into and/or surrounding the support element 112, the material configuration may be coated or jacketed in a manner that retains the desirable range of motion(s) (e.g., flexing, extending, contracting, etc.). FIG. 2A through FIG. 2E illustrate example material structures.

Turning to FIG. 2A, a first example material structure 200 is a braid structure (e.g., braided wire or polymer strands). The braided material, in some examples, may include stainless steel, tungsten, nitinol, nylon, polyether block amide (PEBA), polyurethane (PU), thermoplastic polyurethane (TPU), and/or other medical grade filaments and/or fibers. The strands of the braid structure, in some examples, may include flat, round, and/or ribbon fibers/filaments. In some embodiments, the material structure 200 includes, interior to the braid structure, a flexible liner (e.g., polytetrafluoroethylene (PTFE), PEBA, PU, polyethylene (PE), polyamide, polyether ether ketone (PEEK), fluoropolymer, nylon, etc.). Further, in some embodiments, a flexible outer coating, such as a polymer jacket, may encase an exterior of the braid structure. In embodiments, where the first example material structure 200 includes both a liner and an outer coating, they may be formed of the same or different materials.

As illustrated in FIG. 2B, the first example material structure 200, due to the braid structure, may be designed to expand 202a and compress 202b. When in the compressed state 202b, for example, the first example material structure 200 may be more rigid, providing extra support for an extending element being passed therethrough. Conversely, while in the expanded state 202a, the first example material structure 200 may be more flexible, allowing the first example material structure to traverse complex tortuous pathways while continuing to provide a greater level of rigidity than the extending element passing therethrough.

Turning to FIG. 2C, a second example material structure 210 is a coil structure (e.g., coiled material or a coil of braided strands of material). The coiled material, in some examples, can include a flat, ribbon, or round strand of material. The coil may be formed, in some examples, from stainless steel, nitinol, nylon, PEBA, polyurethane PU, TPU, carbon fiber, PEEK, polyethylene terephthalate (PET), polyethylene naphthalate (PEN), and/or other metal or fiber materials. The coil may be produced, for example, by spiral cutting a rigid or semi-rigid lumen, such as a hypotube. The spiral cut, for example, may transform the rigid or semi-rigid lumen to a flexible coil, where the flexibility depends on the density of the spiral cut (e.g., a number of spirals per unit length or a distance between each pair of spiral cuts). The coiled material, in some embodiments, is coated or jacketed with a flexible material, such as a polymer coating. The flexible material, in further examples, may include PTFE, PEBA, PU, PE, polyamide, PEEK, fluoropolymer, and/or nylon.

As illustrated in FIG. 2D, the second example material structure 210, due to the coil structure, may be designed to stretch 212a and retract 212b. When in the retracted state 212b, for example, the second example material structure 210 may be more rigid, providing extra support for an extending element being passed therethrough. Conversely, while in the expanded state 202a, the first example material structure 200 may be more flexible, allowing the first example material structure to traverse complex tortuous pathways while continuing to provide a greater level of rigidity than the extending element passing therethrough.

FIG. 2E illustrates a third example material structure 220 having a non-uniform coil structure. Rather than a uniform pattern, in some embodiments, a lumen may be cut using a stepped or graduated spiral cutting pattern along its length, where a width of the coil (e.g., distance between cuts) differs along a length of the lumen. As illustrated, from left to right, a segment of an example support element including the third example material structure steps from a narrow (e.g., dense) spiral cut to a wider (e.g., further spaced apart) spiral cut. By applying a stepped spiral cut, for example, a first portion 222a of the third example material structure 220 may be designed to exhibit a first flexibility (e.g., deflection force), while a second portion 222b of the third example material structure 220 is designed to exhibit a second flexibility (e.g., deflection force). Deflection force (e.g., the force required to deflect the section of material to 90° as measured by a force gauge), for example, may be measured in accordance with the Coronary, Peripheral, and Neurovascular Guidewires—Performance Tests and Recommended Labeling guidance document published by the Food and Drug Administration in October 2019. Although the stepped spiral cut is illustrated as including a single step, in other embodiments, the spiral cut may both increase and decrease in density (e.g., stepping and/or graduating towards and away from a segment with greatest flexibility).

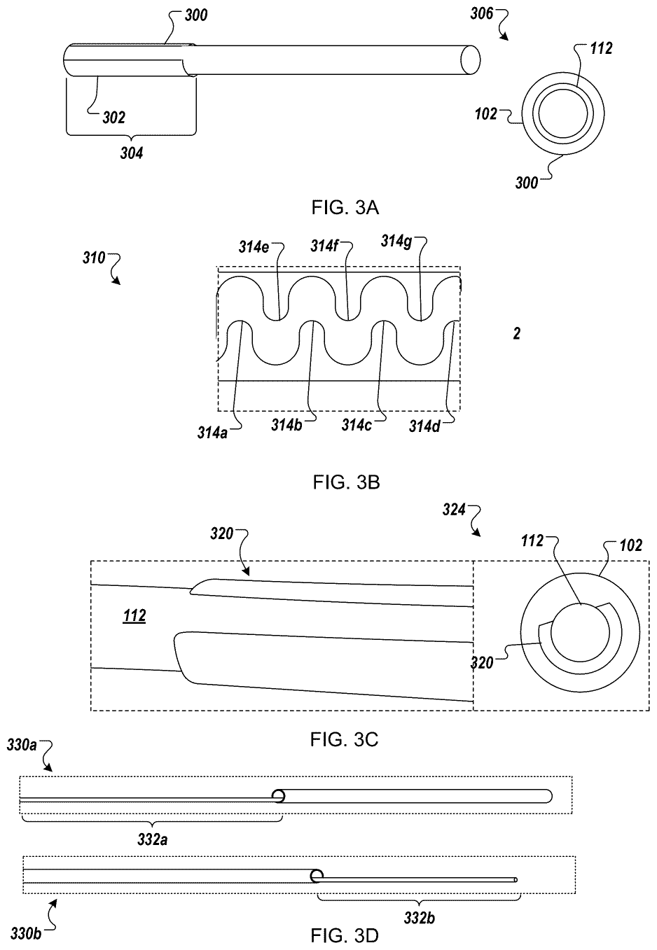

Although described to this point as generally being formed as a lumen having an inner diameter (e.g., a minimum inner diameter in the circumstance of a multiple inner diameter form) for receiving an extending element, a support element, in some embodiments, includes one or more openings along at least a portion of its length. By providing a partially opened support element, in some examples, the support element may be more easily introduced along a length of an extending element, provide greater opportunity for selectively anchoring an extending element, and/or reduce its diameter footprint to better fit within a small diameter catheter while having space for smoothly guiding the extending element through its lumen. FIG. 3A through 3D illustrate example partially open forms for support element structures.

Turning to FIG. 3A, in some implementations, an example support element 300 includes a slit or gap 302 along a first portion 304 (e.g., proximal portion) of a length of the example support element 300. The slit 302, for example may be provided along 5 to 10 cm, 10 to 20 cm, or 20 to 40 cm of the support element 300. In other embodiments, the slit 302 may be provided along an entire length of the support element 300. The slit 302, for example, may be cut along at least a portion of the length of tubular material (e.g., a polymer or metal). In another example, the support element 300 may be formed to include the gap 302 during manufacturing. In some illustrations, an open braided or woven material may be curved to form the first portion 304 of the support element 300, or a coil material may transition to an open zig-zag along the first portion 304 of the support element 300.

As illustrated in a cutaway view 306, the support element 300 may be provided between a catheter (e.g., the guide catheter 102 of FIG. 1A) and a flexible extending element (e.g., the flexible extending element 112 of FIG. 1A). In a further example, in some embodiments, for at least a portion of the length of the support element 300, the support element 300 may surround the flexible extending element and be free of (e.g., extending proximally to or entirely without use of) an exterior element such as a catheter.

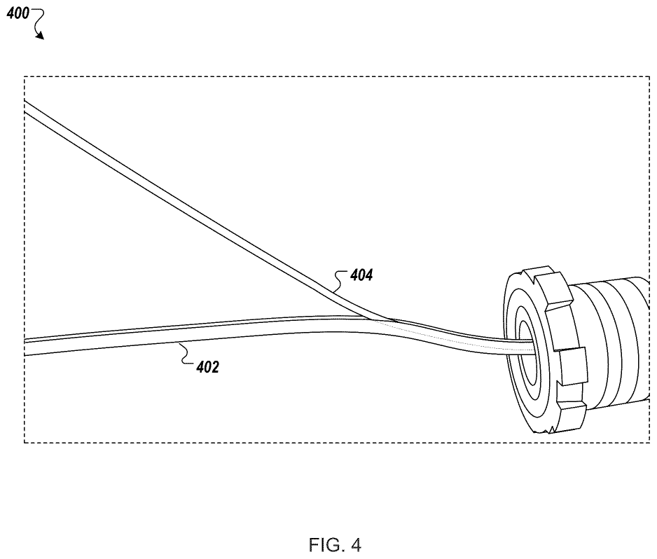

In some embodiments, the slit 302 is provided to allow partial removal of a flexible extending element, such as the flexible extending element 112 of FIG. 1A. For example, as illustrated in FIG. 4, an example use scenario 400 demonstrates a flexible extending element 404 pulled away from its positioning within a support element 402 through a slit or opening within the support element 402. In removing the flexible extending element 404 in this manner, for example, a positioning of the flexible extending element 404 may be maintained relative to the positioning of the support element 402. This may allow, for example, the support element 402 to be advanced down a length of the flexible extending element 404 to a point at which the flexible extending element 404 requires additional rigidity to traverse a challenging segment of anatomy.

Although the slit 302 of the support element 300 is illustrated as being provided straight along a length of the support element 300, in other embodiments, a curved or irregular slit or gap may be provided in a support element. In illustration, turning to FIG. 3B, a partial view of an example support element 310 includes a gap 312 having a wavy pattern that creates extensions (e.g., “fingers”) 314 in the material of the support element 310. The wavy pattern, in other embodiments, may have a more rectangular, or “toothed,” pattern (e.g., with softened or rounded edges). The gap 312 may provide a flexible opening for front-loading (e.g., proximal end loading) of a flexible extending element into the support element 310. Further, the fingers 314 may provide an anchoring mechanism for maintaining the position of the flexible extending element when pulled partially out of the gap 312, as described in relation to FIG. 4. As with the slit 302 of the example support element 300 of FIG. 3A, the gap 312 may be provided along a partial length of the example support element 310 or along the full length.

In some implementations, rather than enclosing fully or mostly around a flexible extending element (e.g., with a limited gap or slit as described above in relation to FIG. 3A and FIG. 3B), a support element may be designed to curve around about a half of a circumference of a flexible extending element (e.g., at least 40%, between 40-50% of the circumference, up to 65% of the circumference, etc.) to provide increased rigidity without substantially enclosing the flexible extending element. In some examples, a partially open support element may provide the benefit of taking up a smaller overall volume of space (e.g., may fit within a smaller instrument catheter and/or be configured to accept a larger flexible extending element than a fully closed support element), allowing for selective rigidity in a desired direction (e.g., through rotating the support element to align with the curvature of the tortuous path), and/or increasing the ease of introducing the support element along a flexible extending element.

Turning to FIG. 3C, for example, an example partially open support element 320 that is configured to partially surround a flexible extending element in a “horseshoe” or “shoehorn” shape, as illustrated in a cutaway view 324. The example partially open support element 320, as illustrated, extends about 55-60% around the circumference of the flexible extending element 112.

In some implementations, only a portion of the length of the partially open support element 320 does not substantially (e.g., except for a slit or gap) or fully surround the flexible extending element. Turning to FIG. 3D, example semi-enclosed support elements 330a and 330b illustrate that a partially surrounding length 332a, 332b may be provided at the proximal end of the support element as with the semi-enclosed support element 330a or at the distal end of the support element as with the semi-enclosed support element 330b. The semi-enclosed support element 330a with the opening at the proximal end, for example, may provide a mechanism for introducing friction (e.g., a manner for locking or retarding movement of one piece of instrumentation in relation to the other). This is illustrated, for example, in FIG. 4. Conversely, the semi-enclosed support element 330b with the opening at the distal end may provide greater “pushability” due to the increased structure at the proximal end of the semi-enclosed support element 330b.

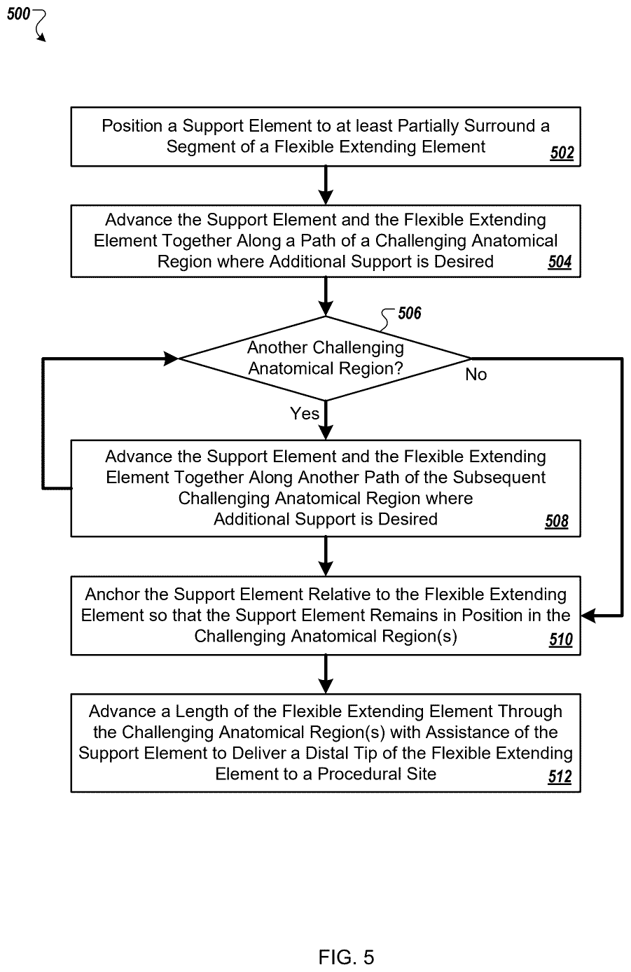

FIG. 5 illustrates a flow chart of an example method 500 for using a support element to assist in navigating an extending element through a tortuous path. The method 500, for example, may be performed using one of the example support elements described above, such as the support element 110 of FIG. 1A, the support element 300 of FIG. 3A, the support element 310 of FIG. 3B, the support element 320 of FIG. 3C, or the support element 330a or 330b of FIG. 3D, along with the flexible extending element 112 of FIG. 1A.

In some implementations, the method 500 begins with positioning a support element to at least partially surround a segment of a flexible extending element (502). The support element may be positioned, in some examples, as illustrated in FIG. 1B, the cutaway view 306 of FIG. 3A, or the cutaway view 324 of FIG. 3C. An external device such as the guide catheter 102 of FIG. 1A and FIG. 1B may at least partially surround an exterior of the support element upon positioning.

In some implementations, the support element and the flexible extending element are advanced together along a path of a challenging anatomical region where additional support is desired (504). The challenging anatomical region, in some examples, can include the aortic arch, a saccular aneurysm, a fusiform aneurysm, or the brachiocephalic veins. The support element, for example, may have a length shorter than or similar to the length of the flexible extending element, such that the support element may be advanced at least so far as to reach known, suspected, or potential challenging anatomical regions.

In other embodiments, rather than being advance together, upon determining that the flexible extending element is becoming caught within a challenging anatomical region, the support element may be advanced to the location and positioned through the challenging path to guide the flexible extending element.

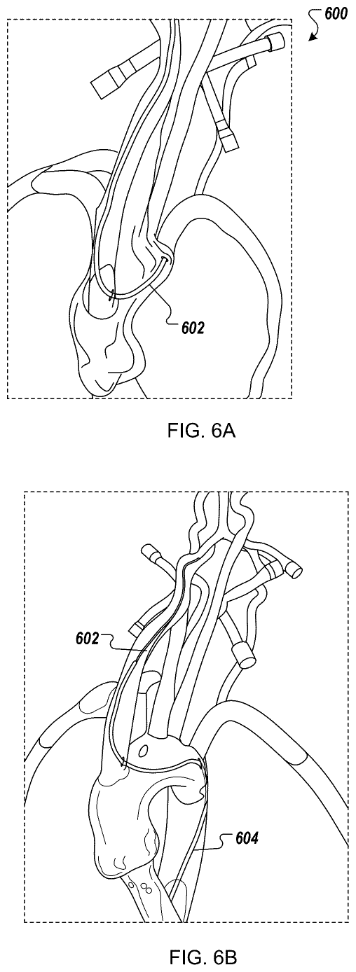

Turning to FIG. 6A, in illustration, an example anatomical region 600 is illustrated with a flexible extending element 602 backed upwards rather than continuing to traverse a downward path. To assist the flexible extending element 602 in proceeding along a desired trajectory, in some embodiments, the flexible extending element 602 is surrounded by a support element 604, as illustrated in FIG. 6B. The support element may be advanced over and/or with the flexible extending element 602 and along a challenging path (e.g., the upward “S” curved path taken by the support element 604 as illustrated in FIG. 6B) to provide the flexible extending element 602 with the rigidity to follow a desired trajectory.

Returning to FIG. 5, in some implementations, if another challenging anatomical region lies along the path to the procedural site (506), the support element is further advanced to the next challenging anatomical region and along the next challenging path to provide additional support (508). As discussed in relation to operation 504, the support element and flexible extending element may be advance together and/or separately.

In some implementations, once the support element is positioned to provide support through one or more challenging anatomical regions, the support element is anchored relative to the flexible extending element so that the support element remains in position in the challenging anatomical region(s) (510). The support element may be anchored, for example, in a manner described in U.S. patent application Ser. No. 18/955,284 filed Nov. 21, 2024 and entitled “Versatile Delivery Catheter,” the contents of which are hereby incorporated by reference in their entirety.

In some implementations, a length of the flexible extending element is advanced through the challenging anatomical region(s) with the assistance of the support element to deliver a distal tip of the flexible extending element to a procedural site (512). As illustrated in relation to FIG. 6B, for example, the flexible extending element 602 is advanced beyond the anchored location of the support element 604.

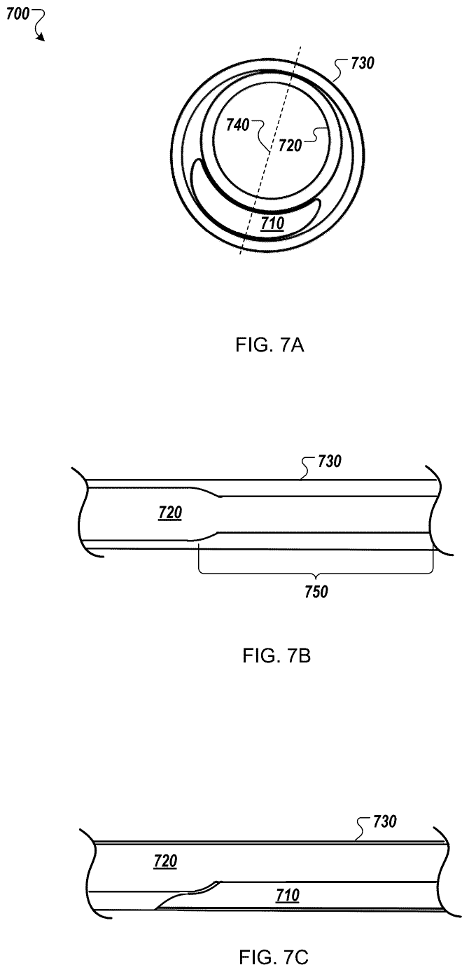

FIG. 7A illustrates an instrument system 700 including a curved support element 710 that is configured to curve against a flexible extending element 720 while limiting its physical footprint by wrapping less than halfway around the diameter of the flexible extending element 720. The example curved support element 710, as illustrated, extends about 27-33% around the circumference of the flexible extending element 112. As illustrated in the cutaway view of FIG. 7A, the combination of the flexible extending element 720 and the curved support element 710 may fit snugly together (e.g., along at least an example diameter 740) within a catheter 730. In this manner, for example, the snug fit within the catheter 730 may encourage the flexible extending element 720 and curved support element 710 to advance together through the vasculature, while inhibiting the flexible extending element 720 from reversing course (e.g., due to pulsatile flow).

Turning to FIG. 7B, in some implementations, the flexible extending element 720 includes a tapered proximal section 750. The tapered proximal section 750 provides for substantial space between the flexible extending element 720 and the catheter 730. Turning to FIG. 7C, the curved support element 710 may have a complementary curvature such that the curved support element 710 creates a snugger fit for the flexible extending element 720 within the catheter 730. The support that the curved support element 710 provides to the tapered proximal section 750 of the flexible extending element 720, for example, may assist in advancing the apparatus to a procedural site and prohibit backing out by the flexible extending element 720. The curved support element 710, for example, includes a thickened portion configured to align with a narrower portion of the flexible extending element 720, followed by a curved portion configured to support a widening curve of the flexible extending element 720. In illustration, the catheter may have a length of up to 130 cm, the flexible extending element may have a length of up to 120 cm, and the curved support element 710 may have a length of up to 80 cm such that it can extend along a length of the tapered proximal section 750 and provide a “shoe horn” gap filling support to the flexible extending element 720, thereby increasing pushability of the apparatus through the catheter 730. As illustrated, the curved support element 710 linearly displaces the flexible extending element 720 within the catheter 730 (e.g., perpendicularly to a longitudinal axis of the catheter 730).

In some implementations, during a medical procedure, a medical professional may wish to track a positioning of an open or partially open support element, such as the support element 300 of FIG. 3A, the support element 310 of FIG. 3B, the support element 320 of FIG. 3C, the support elements 330 of FIG. 3D, and/or the support element 710 of FIG. 7A. Further, the medical professional may wish to track an orientation of an opening of the open or partially open support element. Various radiopaque elements layouts, for example, may assist in tracking both location and orientation within a patient's vasculature.

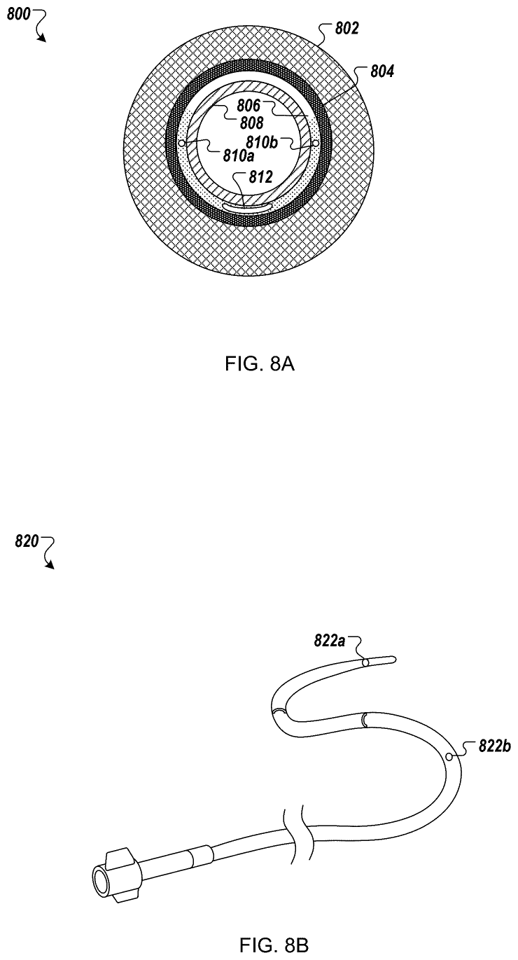

Turning to FIG. 8A, a cross-sectional view 800 of example instrumentation delivery apparatus disposed within a vessel 802 is illustrated. The example instrumentation includes a catheter 804 (e.g., guide catheter, aspiration catheter, or microcatheter, such as the guide catheter 102 of FIG. 1A), an open or partially open support element 806, and a flexible extending element 808 (e.g., such as the flexible extending element 112 of FIG. 1A). Although illustrated as directly abutting, in some embodiments, gaps may exist between various instruments of the example instrumentations, such as between the catheter 804 and the support element 806.

As illustrated, the support element 806 is colored in variegated tones. The variegation is provided to indicate that the opening may differ in size or even vary upon the same support element 806. In some implementations, the support element 806 includes at least two coordinating radiopaque elements configured to identify a position of the support element 806 and an orientation of its opening (e.g., in an upward orientation as illustrated). In the cross-sectional view 800, three radiopaque markers 810a, 810b, and 812 are illustrated, including a set of radiopaque markers 810a and 810b positioned near an upper edge of the opening of the support element 806 and a bottom radiopaque marker 812 indicating a position of the bottom of the support element 806, opposite the opening of the support element 806. The set of radiopaque markers 810a and 810b, as illustrated, are “dot” markers, shown as round beads of radiopaque material. Conversely, the bottom marker is illustrated as a “flat” marker having a wider surface placement. In other embodiments, the radiopaque markers could include different layouts and/or different shapes. In a first example, a single radiopaque marker may be deemed adequate in certain embodiments to mark position and orientation of an open or partially open support element, such as a “bottom” centered marker or a directional marker (e.g., triangle or arrow shaped) that “points” to the opening. In illustration, if using a directional marker, the support element 806 may be rotated until a “point” of the directional marker is recognized. In a second example, a differentiated set of radiopaque markers (e.g., a dot marker on the right side of the opening and a flat marker on the left side of the opening) may be positioned such that an orientation of the opening can be derived based on the relative positioning of the different shapes of the differentiated set of radiopaque markers.

Radiopaque markers, in some implementations, are positioned in multiple locations along an open or partially open support element. Further, the number, shape, and/or positioning of sets of radiopaque markers may differ along a same open or partially open support element. For example, for a partially open support element (e.g., the support elements 330a, 330b of FIG. 3D), a fully surrounding radiopaque band may be placed in one or more positions along the closed length (e.g., fully enclosed lumen) of the partially open (e.g., partially enclosed lumen) support element. FIG. 8B illustrates example radiopaque marker positionings 822a, 822b along a length of an example instrumentation 820.

In some implementations, a flexible extending element, such as a flexible delivery catheter, includes a shaped tip. The shaping of the tip, in some embodiments, is constructed with a gradual reduction (e.g., tapering) in diameter toward a distal end of the flexible extending element to assist in maneuvering through tight and/or curved areas of the vasculature. The tapering may result in a diameter reducing, in some examples, by at least one third, by up to about one half, or by up to about two thirds to initial diameter. In an illustrative example, the tip may taper from a first diameter (e.g., about 0.75 mm) to a second diameter (e.g., about 0.5 mm) over a length of the flexible extending element of between five mm and ten mm. In another example, an angle of a distal region of the flexible extending element may offset from a longitudinal axis of the flexible extending element by over five degrees, up to fifteen degrees, up to thirty degrees, up to forty-five degrees, or under ninety degrees. The curved portion of the flexible extending element may span a length of the flexible extending element of at least five mm or at least ten mm, up to one centimeter, up to three centimeters, up to five centimeters, or up to ten centimeters. In further embodiments, multiple curves may be included, in a wave or “S”-shaped form. In additional embodiments, a curved tip may realign a distal tip of the flexible extending element to point toward a proximal end of the flexible extending element (e.g., over ninety degrees or up to 180 degrees). In using the flexible extending element with a shaped tip, for example, the catheter may facilitate smoother entry into the vasculature, thereby reducing the risk of damaging arterial walls.

In some embodiments, the tip has a curvature where flexibility is enhanced in a particular direction. To retain the curved shape, for example, the tip of the flexible extending element may be manufactured from one or more materials that exhibit shape retention. The materials, in some examples, may include one or more metals, polyurethanes, polyolefins, and/or fluoropolymers.

In some implementations, to assist in maneuvering a curved tip through a tortuous pathway, the tip may include one or more radiopaque markers indicating locations or regions of preformed-curves. The radiopaque marker(s), further to the example, may indicate a direction of each curvature such that the pre-formed curve(s) of the tip may be aligned with curved vascular pathways. The radiopaque marker(s), in one example, may include an arrow or other directional shape indicating a direction in which a pre-formed curve is oriented (e.g., pointing to an inside of the curve or, conversely, an outer side of the curve).

Further, to facilitate smooth traversing through the vasculature, in some embodiments the tapered tip is formed of one or more materials offering a high tensile strength and resistance to kinking or buckling. The tapered tip, for example, may be formed of a material including a nickel titanium alloy (e.g., nitinol).

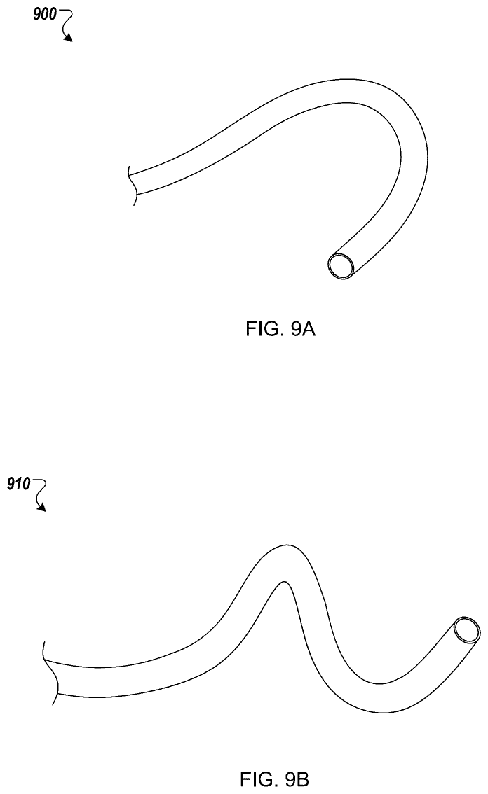

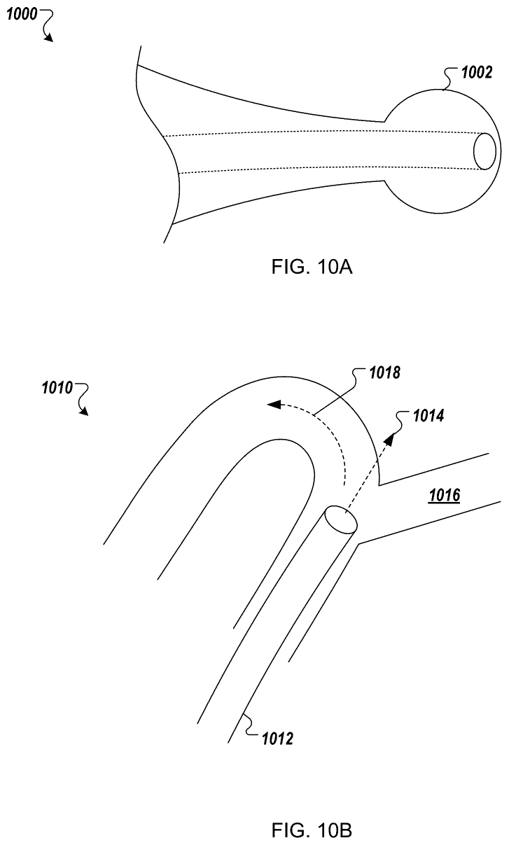

FIG. 9A, FIG. 9B, and FIG. 10A illustrate example curved tip designs. Turning to FIG. 9A, an example “U”-shaped curved tip design 900 for a flexible extending element curves around to point substantially in the direction of the proximate end of the flexible extending element. As described above, the shape of the curved tip may be introduced by including shape retention materials in manufacture. Further, the “U”-shaped curved tip design 900 may include materials selected and/or treated (e.g., by cutting and/or grinding) to flex in the direction of the “U.” In use, the “U”-shaped curved tip design 900 may be maintained within a distal end of a catheter element and/or a support element for introduction into the body and for straight travel. When a branch or bend is encountered, the curved tip design 900 may be extended beyond the catheter element and/or support element to negotiate the curvature and follow the desired path. The “U”-shaped curved tip design 900, for example, may allow for easier traversal of some common procedural pathways, such as the pathway illustrated in FIG. 6A.

Turning to FIG. 9B, an example “S”-shaped curved tip design 910 for a flexible extending element includes an “S” shaping of the flexible extending element leading to the distal end of the flexible extending element. As described above, the shape of the curved tip may be introduced by including shape retention materials in manufacture. Further, the “S”-shaped curved tip design 910 may include materials selected and/or treated (e.g., by cutting and/or grinding) to flex in the directions of the “S.” In use, the “S”-shaped tip design 910 may be maintained within a distal end of a catheter element and/or a support element for introduction into the body and for straight travel. When a tortuous path is encountered, the curved tip design 910 may be extended beyond the catheter element and/or support element to negotiate the curvature and follow the desired path. The “S”-shaped curved tip design 910, for example, may allow easier traversal of some common procedural pathways, such as the pathway illustrated in FIG. 6B.

In further embodiments, a curved tip may include a single curve offset from a longitudinal axis of the flexible extending element by up to ninety degrees. For example, a biased tip design may cause a distal end of the flexible extending element to point in a direction offset from the longitudinal axis of the flexible extending element from one to forty-five degrees. A length of a region of curvature of the flexible extending element, in some examples, may be at least three millimeters, at least five millimeters, or up to ten millimeters.

Turning to FIG. 10A, in some implementations, a “ball”-shaped tip design 1000 is formed to include a bulb 1002 shape at the distal tip. The tip, in some embodiments, includes a tapered region followed by the ball-shaped tip 1000. The ball-shaped tip 1000, for example, may be formed as a solid or hollow ball formed around a cylindrical portion of the flexible extending element. In another example, the ball-shaped tip 1000 may be formed in the material of the flexible extending element itself (e.g., as a widening and narrowing of the flexible extending element. Although illustrated as being substantially round, in other embodiments, the bulbous tip may be oval, tear-shaped, or egg-shaped in form.

The “ball”-shaped tip design 1000 may easily deflect off of side branches to be directed along a desired path. In illustration, turning to an example tortuous pathway diagram 1010 of FIG. 10B, in comparison to a trajectory 1014 of a straight or tapered tip flexible extending element 1012 which may impact with, and potentially damage, branching vasculature, the bulb tip design 1000 of FIG. 10A may instead deflect off of a branch 1016 in the vasculature and follow a desired path 1018.

Although described as separate designs, in some embodiments, a shaped tip of a flexible extending element may include both a ball tip at the distal end and a curvature, such as an S-curvature, leading to the ball tip.

In some embodiments, a catheter includes a segmented liner to enhance flexibility and directional control. The segmentation, for example, may allow the catheter to conform to the natural curves and bends of the vascular system while maintaining structural integrity. The liner, in some examples, may be composed of PTFE, PEBA, PU, PE, PEEK, fluoropolymer, and/or nylon. Segmenting the catheter liner may involve cutting with a blade or a laser along at least a portion of the diameter of the catheter.

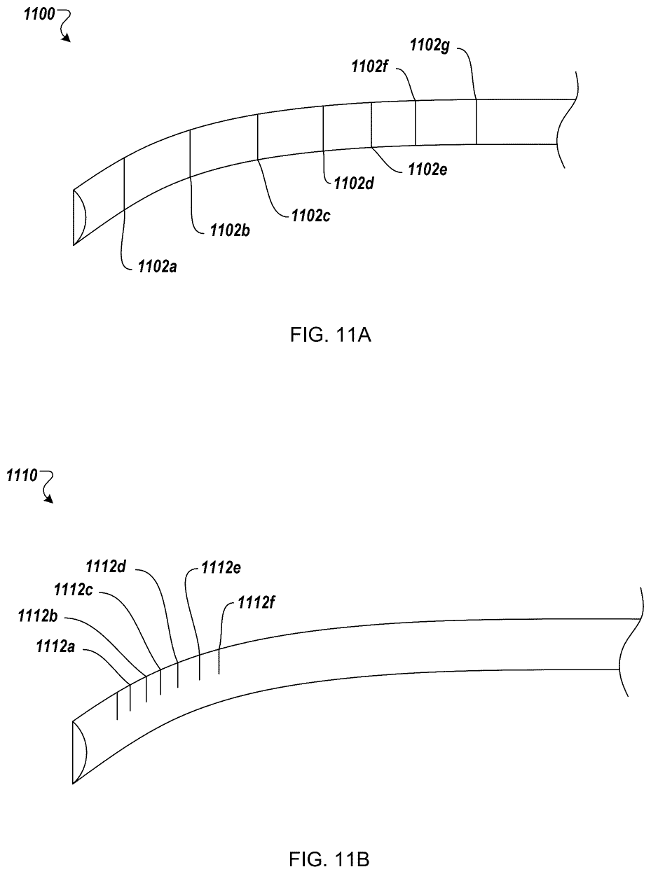

The segmentation, in some embodiments, increases flexibility by cutting consistently around a diameter of the catheter. For example, turning to FIG. 11A, an example catheter liner 1100 includes a spiral cut 1102 along a length of the catheter liner 1100. The spiral cut, in some examples, may be fully segmented (e.g., sliced through the entire way) or partially segmented (e.g., the cuts along the spiral are made with spaces between, such as a dashed line).

In some embodiments, the segmentation is performed to encourage flexibility in a particular direction. For example, the segmentation may involve cuts covering up to half of the diameter of the catheter (e.g., scoring) aligned on an axis parallel to the length of the catheter. The scoring, for example, may be applied horizontally to the axis or on a bias. The cuts may be ground or sliced through at least half of a thickness of the material of the catheter. To encourage directional curvature and/or increased directional flexibility, for example, the cuts may span at least 3 mm, between 3-5 mm, or at least 5 mm. The length of the cut section, in one example, may depend in part on a diameter of the catheter.

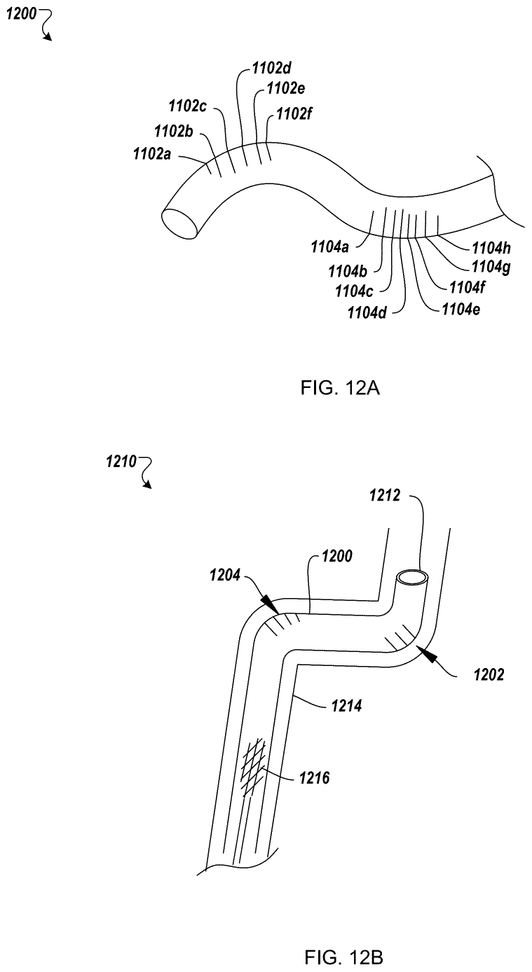

In an illustrative example, turning to FIG. 11B, an example catheter liner 1110 includes a series of cuts 1112a-f. In a further example, different sections of a catheter liner may be cut to encourage flexibility in different directions. Turning to FIG. 12A, an example catheter liner 1200 (e.g., a metal PTFE tube) includes a first series of cuts 1202a-f along a first length of the catheter liner 1200 encouraging the catheter liner 1200 to flex in a first direction and a second series of cuts 1204a-h along a second length of the catheter liner 1200 encouraging the catheter liner 1200 to flex in a second (e.g., approximately opposite) direction.

The embodiments disclosed herein are each designed to improve the efficiency and safety of vascular interventions. For example, segmenting the liner of the catheter to increase flexibility increases precision in manipulation of the catheter, enabling a practitioner to retrieve stents effectively. By applying cuts, scoring, or other segmentation to portions of the flexible extending element, for example, a first length of the flexible extending element may be designed to exhibit a first flexibility (e.g., a higher deflection force), while a second, segmented length may be designed to exhibit a second flexibility (e.g., a lower deflection force). Turning to FIG. 12B, an example deployment scenario 1210 is illustrated in which a catheter 1212 including the example catheter liner 1200 of FIG. 12A is illustrated navigating an artery 1214. As illustrated, the first series of cuts 1202 are aligned with a first bend in the artery 1214 while the second series of cuts 1204 are aligned with a second bend in the artery 1214, thereby reducing strain within the bends of the artery during maneuvering of an instrument 1216, such as a stent retriever instrument, to a treatment site.

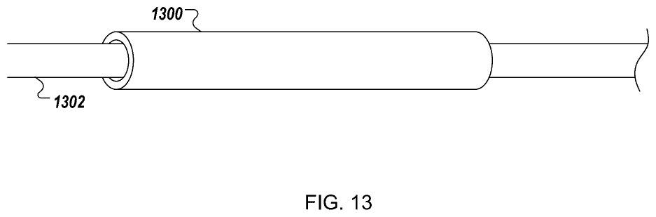

Segmented liners for catheters may be produced and integrated into a catheter in a variety of manners. In some embodiments, turning to FIG. 13, a rod or dowel 1302 is positioned within the length of a catheter liner 1300, and the material of the catheter liner 1300 is cut with a blade (e.g., razor) or laser.

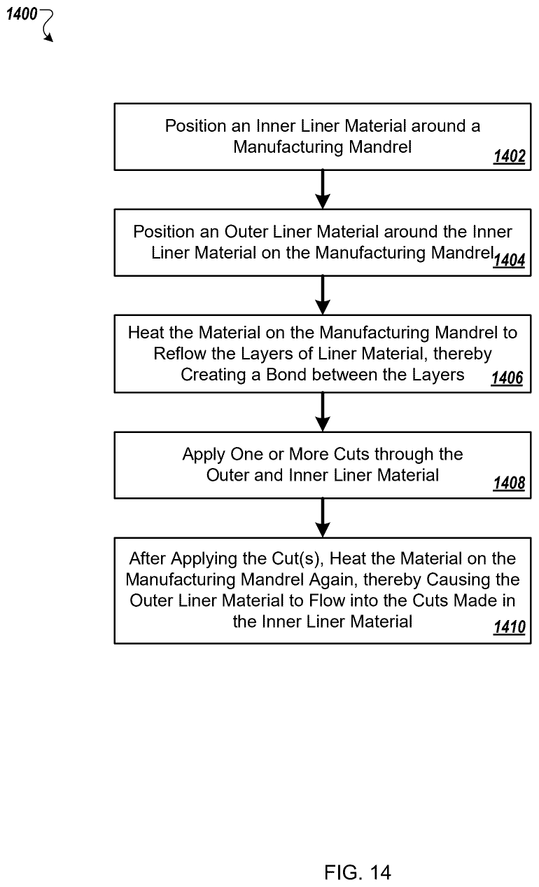

Turning to FIG. 14, a flow chart illustrates an example method 1400 for producing a segmented catheter liner. The method 1400, for example, may be used to produce the segmented liner 1100 of FIG. 11A, the segmented liner 1110 of FIG. 11B, and/or the segmented liner 1200 of FIG. 12A.

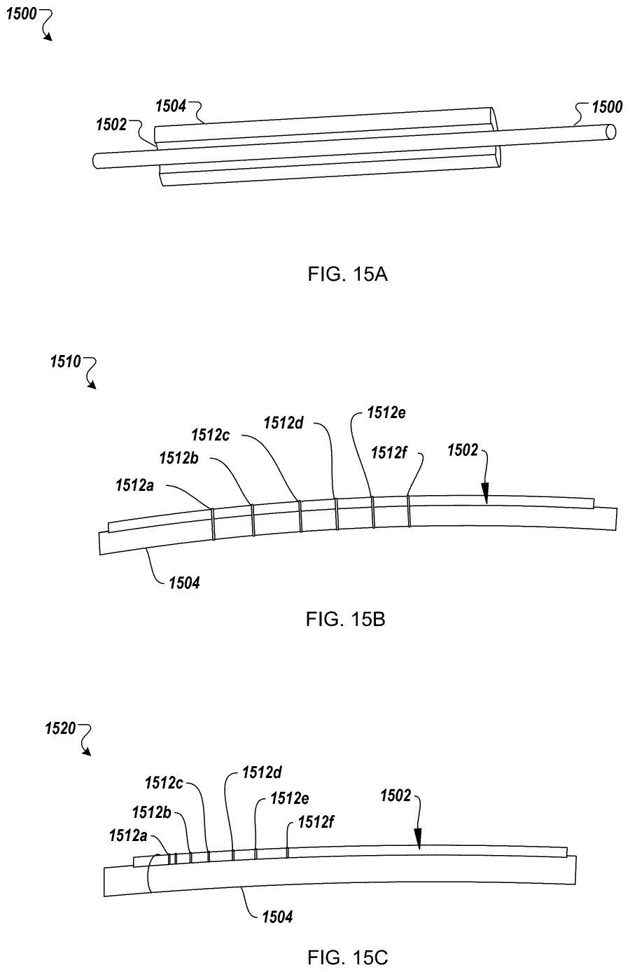

In some implementations, the method 1400 begins with positioning an inner liner material around a manufacturing mandrel (1402). The mandrel may be straight, tapered, or otherwise shaped (e.g., to align with differing outer diameters along a catheter body). The inner liner material, for example, may be slid, in tubular form, onto the manufacturing mandrel, such as the mandrel 1302 of FIG. 13. The inner liner material may be composed of a first material type with a first melt flow index (e.g., temperature at which the material softens and begins to deform). In some examples, the inner liner material may be composed of PTFE, PU, PE, polyamide, PEEK, and/or fluoropolymer. Turning to FIG. 15A, an example cutaway view 1500 of a loaded manufacturing mandrel 1500 illustrates an inner liner material 1502 and an outer liner material 1504.

Returning to FIG. 14, in some implementations, an outer liner material is positioned around the inner liner material on the manufacturing mandrel (1404). The outer liner material, for example, may be slid, in tubular form, over the inner liner material before or after the inner liner material is positioned on the manufacturing mandrel. The outer liner material may be composed of a second material type with a second melt flow index lower than the first melt flow index of the first material type. The outer liner material may be composed of a thermoplastic elastomer such as PEBA and/or nylon.

In some implementations, the layered inner liner material and outer liner material positioned on the manufacturing mandrel may be heated to reheat and reflow the layers of liner material, thereby creating a bond between the materials and softening the materials on the mandrel (1406). Due to a differential between the melt flow index of the inner liner material and the melt flow index of the outer liner material, although displaced from the heat source, the outer liner material may reflow at a similar, or even lower, temperature to the inner liner material.

In some implementations, one or more cuts are applied through the outer and inner liner material (1408). The cuts may be formed perpendicular to or at another angle to the length of the layered liner material. The cuts may be straight, curved, spiral, or another pattern. The cuts may be provided along a portion of the length of the liner material. The density, style, and/or length of the cuts may differ along the length of the liner material. In some examples, the cuts may be applied as illustrated and described in relation to FIG. 11A, FIG. 11B, and FIG. 12A. As illustrated in an example cutaway view 1510 of FIG. 15B, for example, a series of cuts 1512 extend through both the outer liner material 1504 and the inner liner material 1502.

In some implementations, after applying the one or more cuts (1408), the layers of liner material are reflowed on the manufacturing mandrel, thereby flowing the outer liner material into the cuts made through the inner liner material (1410). In this manner, the cuts may be partially or fully filled with the material of the outer liner, avoiding any pinching or snagging of instrumentation due to the texture of the cuts, while retaining the increased flexibility provided by the segmentation. For example, as illustrated in a cutaway view 1520 of a FIG. 15C, the outer liner material 1504, after reflowing, is now solid, while the inner liner material 1502 continues to demonstrate segmentation as shown by the cut lines 1512a-f. The cut lines 1512a-f, for example, may be at least partially filled with the material of the outer liner 1504 while imparting increased flexibility to the inner liner material 1502.

Although described in an example order of operations, in other embodiments, certain operations of the method 1400 may be performed in a different order. For example, as described above, the inner liner material may be inserted within the outer liner material prior to positioning the material on the manufacturing mandrel (1402). Further, although described as a particular set of operations, in other embodiments, the method 1400 may include more or fewer operations. In illustration, in some embodiments, the inner liner material may not be heated to a point of flow, such that the outer liner material bonds to the inner liner material without flow of the inner liner material. In another illustrative example, the bonded layers of liner material may be removed from the manufacturing mandrel prior to the cutting process (1408), then replaced for the second heating operations (1410). Other modifications of the method 1400 are possible.

Further, the catheter may be treated with a specialized coating to improve lubrication within the lumen of the catheter, thereby facilitating passage of stent retrieval devices. A segmented liner, in some embodiments, is coated to enhance lubricity, reducing friction and improving the ease of insertion and maneuverability of stent retrieval apparatus. The specialized coating, for example, may be formulated to be biocompatible and durable, resisting degradation during use. The specialized coating may be hydrophilic or hydrophobic, depending upon the procedural function desired for the catheter.

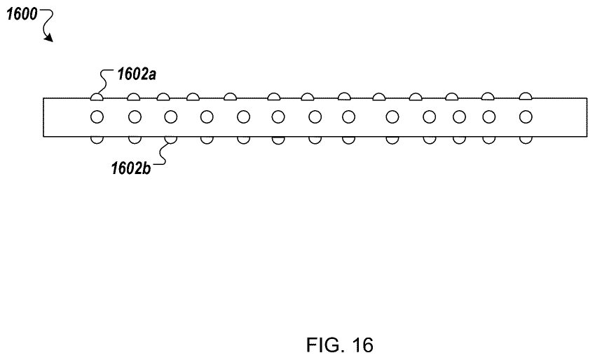

In some implementations, the surface area of an innermost diameter of a catheter is reduced, contributing to its lubricity. Responsive to surface area reduction, a corresponding reduction in friction with instrumentation provided within the catheter is achieved. The cross-sectional profile of the catheter, for example, may be optimized by incorporating textured or coil structures designed to minimize contact between instrumentation and the catheter walls. As illustrated in a cross-sectional view of an inner liner of an example catheter 1600 of FIG. 16, a textured structure may produce a raised surface 1602 around the inner liner. The irregular surface 1602 on the inner diameter of the example catheter liner 1600 provides a reduced contact area for instrumentation fed within the catheter, such as a stent retriever, flexible extending element, and/or support element described above. Although shown with pronounced “bumps” for ease of illustration, in practice, the texture may be a lightly textured (e.g., bumpy, ridged, etc.) surface. A pre-textured manufacturing mandrel may be used, in illustration, to impart a surface texture to the inner surface of the catheter liner through heating and flowing the inner surface material to conform with the pre-textured surface (e.g., during the heating operation 1410 of FIG. 14). In another illustrative example, a material component may be introduced to create a micro textured surface. The material component (e.g., a dust or finely crushed material), such as barium sulfate powder or iron powder, can be embedded in the inner diameter of the catheter liner to produce a micro-level texture to the internal surface of the catheter. In an illustrative example, a magnetic material component may be applied via electrostatic texturization, where an electromagnetically charged manufacturing mandrel is dusted with the magnetic material component and, upon reflowing the inner catheter liner material, the electromagnet may be deactivated, thereby transferring the magnetic material component to the surface area of the inner liner. By minimizing contact with the catheter walls, the instrumentation, such as a stent retriever, can navigate more easily through the catheter.

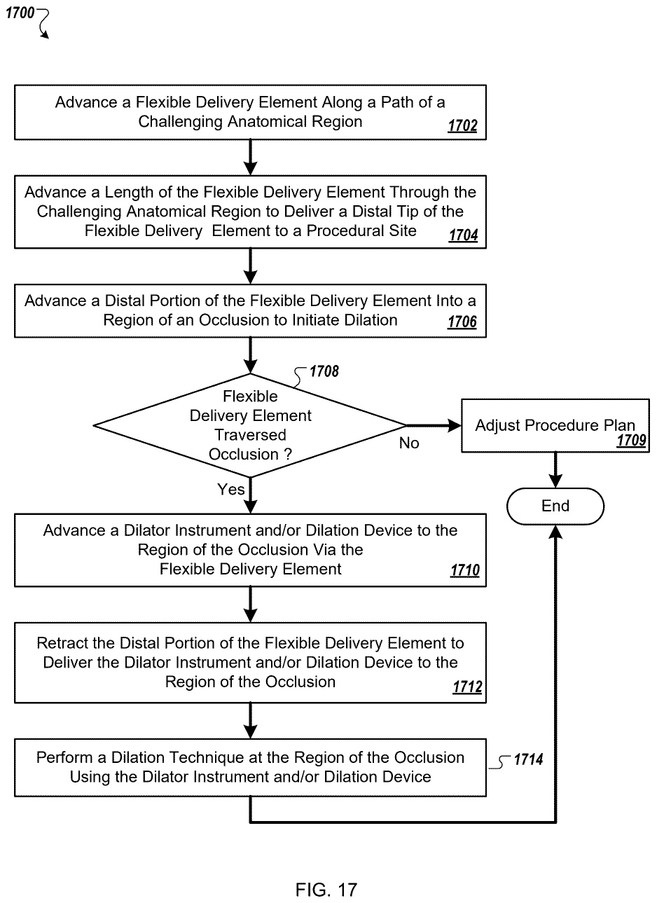

Turning to FIG. 17, a flow chart of an example method 1700 for dilating an occlusion is illustrated. The method 1700, for example, may be performed in combination with various instruments disclosed herein, such as a flexible microcatheter device.

In some implementations, the method 1700 begins with advancing a flexible delivery element along a path of a challenging anatomical region (1702). The flexible delivery element, in some examples, may be a catheter, microcatheter, or delivery catheter. The flexible delivery element may include features of the flexible extending element 112 of FIG. 1A and FIG. 1B. The flexible delivery element may include one or more material structures such as the material structure 200 of FIG. 2A and/or the material structure 210 of FIG. 2C. The flexible delivery element may further include a shaped and/or tapered tip design, such as the curved tip design 900 of FIG. 9A, the curved tip design 910 of FIG. 9B, and/or the shaped tip design 1000 of FIG. 10A. In some embodiments, the flexible delivery element includes a segmentation pattern for increased flexibility, such as the segmentation examples illustrated in FIG. 11A, FIG. 11B, and FIG. 12A. Further, the flexible delivery element may include a texturized surface as described, for example, in relation to FIG. 16. The challenging anatomical region, in some examples, may include “U”- or “S”-shaped bends, such as the paths illustrated in FIG. 6A and FIG. 6B. As described in relation to FIG. 1A and FIG. 1B, for example, the challenging anatomical region may include various neurovascular, cardiovascular, and/or peripheral access paths such as, in some examples, the aortic arch, a saccular aneurysm, a fusiform aneurysm, or the brachiocephalic veins.

In some implementations, a length of the flexible delivery element is advanced through the challenging anatomical region to delivery a distal tip of the flexible delivery element to a procedural site (1704). The flexible delivery element may be advanced through the challenging anatomical region, for example, using techniques described in relation to the method 500 of FIG. 5. The procedural site may be a site of a chronic occlusion, such as a spasmed or constricted artery, an intracranial stenosis, or a calcified lesion. In further examples, the procedure site may include a thrombus or an atheroma (e.g., plaque build-up).

In some implementations, the distal portion of the flexible delivery element is advanced into a region of an occlusion to initiate dilation (1706). The distal portion of the flexible delivery element, for example, may be used to gently provide an opening into an occluded area within the vasculature. The flexible delivery element, in this circumstance, may be used in lieu of an arterial dilator or balloon, for example.

The distal portion of the flexible delivery element, in some embodiments, includes a tip design configured to traverse an occluded pathway. For example, the tip may be biased to point slightly off center at a pre-formed angle (e.g., up to forty-five degrees offset from a longitudinal axis of the flexible delivery element) such that the tip is configured to be off-center from a central point of an occlusion. The curvature of the tip, for example, may be formed in a manner described above.

If the flexible delivery element fails to traverse the occlusion (1708), in some implementations, the practitioner may adjust the procedure plan (1709). For example, a different instrumentation, such as a thicker flexible delivery element, may be provided. In another example, a pharmacological agent may be administered, followed by continuing (1706) to attempt to traverse.

Once the occlusion has been traversed by the flexible delivery element (1708), in some implementations, a dilator instrument and/or dilation device is advanced to the region of the occlusion via the flexible delivery element (1710). Dilator instruments, in some examples, can include a vascular dilator, an intravascular lithotripsy (IVL) device, and/or an angioplasty balloon. In some examples, dilator devices may include a stent or a bioabsorbable scaffold. The dilator instrument and/or dilation device may have been advanced, for example, concurrently with the flexible delivery element such that it has been queued up while the distal tip is advanced separately to initiate dilation (1706). In another example, upon successful positioning of the flexible delivery element, the dilator instrument and/or dilation device may be introduced within the flexible delivery element for advancement. In the illustration of FIG. 12B, for example, the instrument 1216 is introduced within the flexible element 1212.

In some embodiments, as illustrated in relation to the apparatus 100 of FIG. 1A, a guide catheter, the delivery element, and a guide wire may be used to advance the delivery element to the region of the occlusion. After traversing the occlusion, the flexible delivery element may be removed from the guide catheter, and a dilator instrument and/or dilation device positioning instrument may be advanced to the region of the occlusion along the guide wire. Related U.S. Patent Application Publication No. 2005/0161630 entitled “Versatile Delivery Catheter,” for example, describes example mechanisms for locking a position of a guide wire while replacing instrumentation at a procedure site. A similar order of operations, in a further illustration, may be performed if the flexible delivery element itself failed to traverse the occlusion (1708). For example, a dilation device may be attempted if the flexible delivery element fails to traverse the occlusion.

In some implementations where the dilator instrument and/or the dilation device are delivered via the flexible delivery element, the distal tip of the flexible delivery element is retracted to deliver the dilator instrument and/or the dilation device to the region of the occlusion (1712). For example, the distal tip of the flexible delivery element may be removed from the region of the occlusion by drawing back manually or robotically. In illustration, the dilator instrument may be a stent or a bioabsorbable scaffold, and retracting the distal tip may result in deploying the stent or bioabsorbable scaffold at the location of the occlusion. In this manner, the flexible delivery element may enable a single pass dilation (via the flexible delivery element itself) and stent or bioabsorbable scaffold delivery procedure, allowing stent/bioabsorbable scaffold deployment in a quick and simple manner. Further, using only the flexible delivery element allows for a potentially lower procedural risk.

In some implementations, a dilation technique is performed at the region of the occlusion using the dilator instrument and/or the dilator device (1714). The dilation technique, in some examples, can include stent delivery, balloon angioplasty, IVL, bioabsorbable scaffold delivery, and/or further dilation using a vascular dilator.

Although described in relation to a particular set of operations, in some embodiments, the method 1700 includes more or fewer operations. For example, as discussed above, retracting the distal tip (1712) may result in deployment of a stent or bioabsorbable scaffold, such that no separate dilation technique (1714) is performed. In certain embodiments, the advancing of the flexible delivery element may be performed in lieu of a traditional Dotter technique. For example, rather than a guide wire followed by a catheter as used in the Dotter technique, the flexible delivery element may initially traverse the occlusion, allowing for dilation via a single pass. Further, although described in relation to a particular series of operations, in other embodiments, certain operations may be performed in a different order and/or concurrently. For example, the dilator instrument and/or dilation device may be advanced within the flexible delivery element (1710) at least partially concurrently with advancing the distal tip of the flexible delivery element to the region of the occlusion (1706). Other modifications to the method 1700 are possible.

While certain embodiments have been described, these embodiments have been presented by way of example only and are not intended to limit the scope of the present disclosure. Indeed, the novel methods, apparatuses and systems described herein can be embodied in a variety of other forms; further, various omissions, substitutions and/or changes in the form of the methods, apparatuses and systems described herein can be made without departing from the spirit of the present disclosure. The accompanying claims and their equivalents are intended to cover such forms or modifications as would fall within the scope and spirit of the present disclosure.

1. A support instrument configured to provide additional stiffness to a delivery instrument during advancement of the delivery instrument to a procedural site, the support instrument comprising:
a shaft defining, along at least a portion of a length of the shaft, a partially enclosed lumen configured to receive at least a portion of the delivery instrument, wherein
a distal portion of the shaft comprises a support instrument deflection force greater than a delivery instrument deflection force of a distal portion of the delivery instrument, and
the partially enclosed lumen is configured to enable advancement of the delivery instrument while the support instrument remains in a same position or is withdrawn; and
at least one radiopaque marker disposed along the length of the shaft, wherein the at least one radiopaque marker is configured to identify an orientation of an opening of the partially enclosed lumen.

2. The support instrument of claim 1, wherein the opening of the partially enclosed lumen defines a uniform gap.

3. The support instrument of claim 1, wherein the shaft defines, along a second portion of the length of the shaft, a fully enclosed lumen configured to receive another portion of the delivery instrument.

4. The support instrument of claim 1, wherein at least a distal portion of the shaft comprises a flexible structure formed from medical grade filaments or fibers.

5. The support instrument of claim 1, wherein a bottom radiopaque marker of the at least one radiopaque marker is disposed on a surface of the shaft opposite the opening.

6. The support instrument of claim 1, wherein the opening is configured, at least at a proximal end of the shaft, to allow a section of the delivery instrument to be withdrawn at an angle to the length of the shaft, thereby locking a position of the delivery instrument relative to the support instrument.

7. The support instrument of claim 1, wherein the opening comprises a wavy or toothed pattern.

8. The support instrument of claim 1, wherein a material composition of the support instrument comprises one or more of a hypotube, stainless steel, nitinol, or carbon fiber.

9. The support instrument of claim 1, wherein a material composition of the support instrument comprises one or more of nylon, polyether block amide (PEBA), polyurethane (PU), thermoplastic polyurethane (TPU), polyether ether ketone (PEEK), polyethylene terephthalate (PET), or polyethylene naphthalate (PEN).

10. The support instrument of claim 1, wherein the support instrument comprises a tapered distal tip.

11. A method for using a support element to assist in navigating an extending element through a tortuous path, the method comprising:
providing the support element comprising a shaft;
positioning at least a segment of the extending element along an inner curvature of the shaft of the support element;
advancing the support element and the extending element together along the tortuous path to align a section of the support element with the tortuous path; and
continuing to advance the extending element through the tortuous path to deliver a distal portion of the extending element beyond a distal end of the support element through a section of vasculature distal to the tortuous path.

12. The method of claim 11, wherein positioning the support element comprises positioning the support element within a catheter and extending along at least a portion of a length of the catheter.

13. The method of claim 12, wherein
the extending element comprises a tapered section spanning a first length of the extending element; and
the support element comprises a complementary thickened section spanning a second length of the support element shorter than or a same length as the first length;
wherein, upon positioning the support element within the catheter, the thickened section of the support element aligns with at least a section of the first length of the extending element, thereby linearly displacing the first length of the extending element to align closer to a wall of the catheter.

14. The method of claim 11, wherein the support element defines, along portions of a length of the shaft, at least one of a partially enclosed lumen or a fully enclosed lumen.

15. The method of claim 11, further comprising, prior to continuing to advance the extending element, anchoring the support element relative to the extending element to maintain a current position of the support element.

16. The method of claim 11, wherein:
the support element comprises, along portions of a length of the shaft, at least one radiopaque element; and
aligning the section of the support element with the tortuous path comprises aligning the at least one radiopaque element with the tortuous path.

17-60. (canceled)
